(19) United States
(12) Patent Application Publication
(10) Pub. No.: US 2026/0338693 A1
(43) Pub. Date: Oct. 29, 2026

(54) METHODS FOR RAPID READING AND ANALYSIS OF FLUORESCENCE IMMUNOCHROMATOGRAPHIC TEST STRIPS BASED ON DEEP LEARNING

(71) Applicant: NANCHANG UNIVERSITY, Nanchang (CN)

(72) Inventors: Xiaolin HUANG, Nanchang (CN); Weitao HE, Nanchang (CN); Yonghua XIONG, Nanchang (CN); Lu DING, Nanchang (CN); Yuankui LENG, Nanchang (CN); Xirui CHEN, Nanchang (CN); Yuhao WU, Nanchang (CN)

(73) Assignee: NANCHANG UNIVERSITY, Nanchang (CN)

(21) Appl. No.: 19/648,147

(22) Filed: Apr. 15, 2026

(30) Foreign Application Priority Data
Apr. 29, 2025 (CN) 202510550644.6

(51) Int. Cl. G01N 21/64; A61M 5/168; G01N 21/77; G01N 33/543; G16H 10/40

(52) U.S. Cl. CPC G01N 21/6428; A61M 5/16804; G01N 21/77; G01N 33/54387; G16H 10/40; G01N 2021/7759

(57) ABSTRACT

Provided is a method for rapid reading and analysis of a fluorescence immunochromatographic test strip based on deep learning, including: obtaining fluorescence spectrum data of a sample to be tested on the fluorescence immunochromatographic test strip; constructing a signal preprocessing model; constructing a signal feature extraction model; constructing a time series prediction model; constructing a detection result output model; and inputting collected fluorescence intensity signals of the reaction in progress into the signal preprocessing model for time-domain distinguishing and labeling, extracting a plurality of fluorescence intensity signal feature vectors through the signal feature extraction model, predicting a reaction-complete feature vector through the time series prediction model, and obtaining a detection result of an analyte in the sample to be tested through the detection result output model.

### CROSS-REFERENCE TO RELATED APPLICATIONS

This application claims priority to Chinese Application No. 202510550644.6, filed on Apr. 29, 2025, the entire contents of which are incorporated herein by reference.

### TECHNICAL FIELD

The present disclosure relates to the field of fluorescence immunochromatographic technology, and specifically relates to a method for rapid reading and analysis of a fluorescence immunochromatographic test strip based on deep learning.

### BACKGROUND

After an acute myocardial infarction (AMI) occurs, myocardial cells are damaged, causing cardiac troponin I (cTnI) to be released from the cells into the bloodstream. The release can usually be detected within a few hours after myocardial injury, so it can serve as an early biomarker for early diagnosis and identification of acute myocardial infarction. According to the “Guidelines for the Diagnosis and Treatment of Acute Myocardial Infarction”, the emergency department should strive to complete clinical examinations for patients suspected of having AMI within 10 minutes, and for eligible patients, thrombolytic therapy should be initiated within 30 minutes after the visit or direct emergency percutaneous coronary intervention should be initiated within 90 minutes. Clinically, test strips are widely used for the detection of cTnI. The fluorescence immunochromatographic test strip has the characteristics of high sensitivity and strong quantitative analysis capability, but the test strip usually requires a sample addition reaction time of 8-15 minutes. Shortening the detection time can provide patients with more opportunities for treatment and rescue.

Machine learning, especially deep learning, can handle complex nonlinear relationships. When there are complex and variable dependencies among input data, it can capture high-dimensional features in the data through learning. Machine learning has been widely applied in multiple medical fields, especially in image analysis, disease prediction, genomics, and the processing of biological signals. The present disclosure innovatively utilizes a machine learning algorithm during the test strip reading process to predict the growth trend of the fluorescence intensity signal through time series data, thereby shortening the diagnosis time for myocardial infarction.

### SUMMARY

One or more embodiments of the present disclosure provide a method for rapid reading and analysis of a fluorescence immunochromatographic test strip based on deep learning, which can solve the technical problem in the prior art of a long sample addition reaction time of the fluorescence immunochromatographic test strip that is detrimental to rapid detection.

One or more embodiments of the present disclosure provide a method for rapid reading and analysis of a fluorescence immunochromatographic test strip based on deep learning. The method includes: obtaining fluorescence spectrum data of a sample to be tested on the fluorescence immunochromatographic test strip; constructing a signal preprocessing model to distinguish and label a reaction stage of the fluorescence spectrum data to obtain labeled fluorescence spectrum data, wherein the reaction stage includes reaction not started, reaction in progress, and reaction completed; constructing a signal feature extraction model to extract a fluorescence intensity signal feature vector from the labeled fluorescence spectrum data; constructing a time series prediction model to perform time series prediction processing on a reaction-in-progress feature vector to obtain a reaction-complete feature vector; wherein constructing the time series prediction model includes: combining a plurality of fluorescence intensity signal feature vectors into a feature vector; setting a time window to obtain a feature vector sequence of a fixed length; sliding the time window to obtain a plurality of feature vector sequences covering an entire reaction stage, and obtaining training samples; and inputting the training samples into a Transformer model to construct the time series prediction model; constructing a detection result output model to perform a diagnosis based on the reaction-complete feature vector to obtain a detection result of an analyte in the sample to be tested; and inputting collected fluorescence intensity signals of the reaction in progress into the signal preprocessing model for time-domain distinguishing and labeling, extracting the plurality of fluorescence intensity signal feature vectors through the signal feature extraction model, predicting the reaction-complete feature vector through the time series prediction model, and obtaining the detection result of the analyte in the sample to be tested through the detection result output model.

### BRIEF DESCRIPTION OF THE DRAWINGS

FIG. 1 is a flowchart illustrating an exemplary process for rapid reading and analysis of a fluorescence immunochromatographic test strip based on deep learning according to some embodiments of the present disclosure;

FIG. 2 is a flowchart illustrating another exemplary process for rapid reading and analysis of a fluorescence immunochromatographic test strip based on deep learning according to some other embodiments of the present disclosure;

FIG. 3 is a schematic diagram illustrating typical fluorescence signal spectrums of a fluorescence immunochromatographic method according to some embodiments of the present disclosure, where (a) of FIG. 3 is a fluorescence intensity change diagram of the fluorescence immunochromatographic method for a positive sample, (b) of FIG. 3 is a fluorescence intensity change diagram of the fluorescence immunochromatographic method for a weak positive sample, and (c) of FIG. 3 is a fluorescence intensity change diagram of the fluorescence immunochromatographic method for a negative sample;

FIG. 4 is a schematic diagram illustrating a network structure of a signal preprocessing model according to some embodiments of the present disclosure;

FIG. 5 is a schematic diagram illustrating a network structure of a signal feature extraction model according to some embodiments of the present disclosure;

FIG. 6 is a schematic diagram illustrating a network structure of a time series prediction model according to some embodiments of the present disclosure; and

FIG. 7 is a schematic diagram illustrating a network structure of a detection result output model according to some embodiments of the present disclosure.

### DETAILED DESCRIPTION

In order to enable those skilled in the art to better understand the solutions of the present disclosure, the technical solutions in the embodiments of the present disclosure will be clearly and completely described below with reference to the accompanying drawings of the embodiments of the present disclosure. Obviously, the described embodiments are merely a portion of the embodiments of the present disclosure, and not all of the embodiments. Based on the embodiments in the present disclosure, all other embodiments obtained by one of ordinary skill in the art without making inventive efforts fall within the protection scope of the present disclosure.

The terms “comprise” and “include” and any variations thereof in the present disclosure, the claims, and the above-mentioned accompanying drawings are intended to cover non-exclusive inclusion. For example, a process, a method, a system, a product, or a device comprising a series of steps or units is not limited to the listed steps or units, but may optionally also include unlisted steps or units, or may optionally also include other steps or units inherent to these processes, methods, products, or devices. The descriptions of the terms “first”, “second”, and “third”, and so on, are used to distinguish different objects, etc., which do not represent sequential order, and do not limit the “first”, “second”, and “third” to be of different types.

In the description of the embodiments of the present disclosure, “exemplary”, “for example”, or “by way of example” and so on, are used to indicate examples, illustrations, or descriptions. Any embodiment or design scheme described as “exemplary”, “for example”, or “by way of example” in the embodiments of the present disclosure should not be construed as being more preferred or advantageous than other embodiments or design schemes. Rather, the words “exemplary”, “for example”, or “by way of example” and so on, are intended to present relevant concepts in a concrete manner.

In the description of the embodiments of the present disclosure, unless otherwise specified, “/” means “or”. For example, A/B can mean A or B. The “and/or” in the text is merely a relational term describing associated objects, indicating that there may be three relationships. For example, A and/or B can mean: A exists alone, A and B coexist, or B exists alone. In addition, in the description of the embodiments of the present disclosure, “a plurality of” refers to two or more.

In some processes described in the embodiments of the present disclosure, a plurality of operations or steps appearing in a specific order are included. However, it should be understood that these operations or steps may not be performed in the order in which they appear in the embodiments of the present disclosure or may be performed in parallel. The sequence numbers of the operations are merely used to distinguish different operations, and the sequence numbers themselves do not represent any execution order. In addition, these processes may include more or fewer operations, and these operations or steps may be performed sequentially or in parallel, and these operations or steps may be combined.

In order to make the objectives, technical solutions, and advantages of the present disclosure clearer, the embodiments of the present disclosure will be further described in detail below with reference to the accompanying drawings.

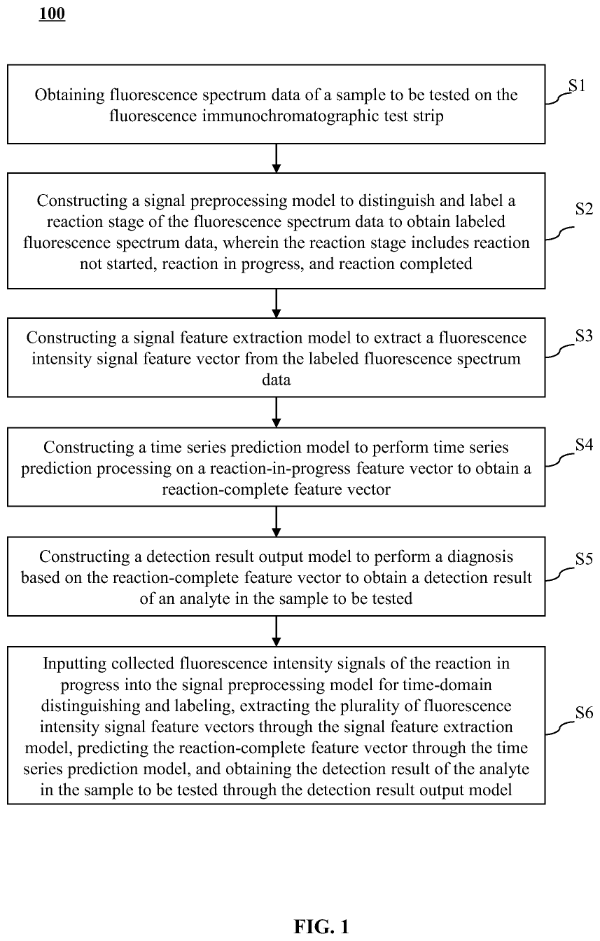

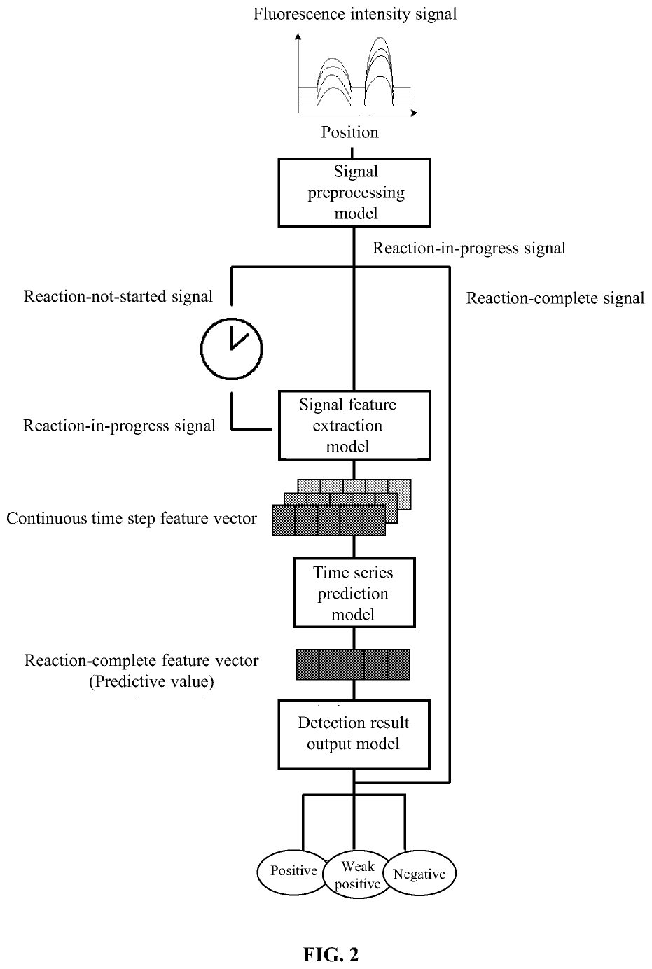

FIG. 1 is a flowchart illustrating an exemplary process for rapid reading and analysis of a fluorescence immunochromatographic test strip based on deep learning according to some embodiments of the present disclosure. FIG. 2 is a flowchart illustrating another exemplary process for rapid reading and analysis of a fluorescence immunochromatographic test strip based on deep learning according to some other embodiments of the present disclosure.

In some embodiments, as shown in FIGS. 1 and 2, the process for rapid reading and analysis of the fluorescence immunochromatographic test strip based on deep learning (hereinafter referred to as the process 100) includes the following steps S1 to S6. In some embodiments, the steps S1 to S6 may be performed by a processor.

In some embodiments, the processor may include a general-purpose processor, a digital signal processor (DSP), an application-specific integrated circuit (ASIC), a field-programmable gate array (FPGA), or other programmable logic devices, discrete gate or transistor logic devices, or discrete hardware components. The general-purpose processor may be a microprocessor, or the processor may also be any conventional processor, etc.

Step S1, obtaining fluorescence spectrum data of a sample to be tested on the fluorescence immunochromatographic test strip.

The fluorescence immunochromatographic test strip may also be referred to as a test strip.

The sample to be tested refers to a sample that includes a biological marker to be detected (i.e., an analyte). For example, in the diagnosis of acute myocardial infarction (AMI), the sample to be tested may include a liquid sample, such as a blood sample, and the analyte may be cTnI, etc.

The fluorescence spectrum data refers to a data set reflecting fluorescence intensity signals at different positions on the test strip. In some embodiments, the fluorescence spectrum data includes fluorescence intensity signals (also referred to as fluorescence intensities) at a plurality of positions. The fluorescence spectrum data may be a series of data generated by a reader scanning the test strip once at a preset frequency (e.g., once every 30 seconds). Each set of data represents fluorescence intensity signals at a plurality of positions within the full length of the test strip at a certain time point.

The fluorescence immunochromatographic test strip has a detection line and a control line. The detection line (also referred to as the T-line) is used to detect whether there is an analyte in the sample to be tested. If the analyte exists in the sample to be tested, a fluorescence intensity signal may be collected at the detection line. The control line (also referred to as the C-line) is used to determine whether the test strip itself is valid. Regardless of the presence or absence of the analyte in the sample to be tested, a fluorescence intensity signal may also be collected at the control line.

In some embodiments, the fluorescence spectrum data at a single time point may be presented as a waveform curve where the fluorescence intensity signal changes with position. The fluorescence intensity signals near the T-line and the C-line may form feature peaks, which are referred to as a T-line peak and a C-line peak.

In some embodiments, obtaining of the fluorescence spectrum data of the sample to be tested on the fluorescence immunochromatographic test strip may be implemented by the processor through the reader, specifically including: dropping the sample to be tested onto the fluorescence immunochromatographic test strip, reading the fluorescence intensity signals at each position on the fluorescence immunochromatographic test strip at a preset frequency by the reader until a reaction is completed, and obtaining the fluorescence spectrum data of the entire reaction stage. The reader sends the fluorescence spectrum data to the processor.

In some embodiments of the present disclosure, by reading fluorescence intensity signals at a preset frequency through the reader, a reaction line can be precisely located, signal changes can be dynamically quantified, and multi-dimensional features can be extracted, thereby providing high-resolution spatio-temporal data for a machine learning model, to realize rapid and high-precision early predictive detection.

Exemplarily, 75 L of a blood sample from a patient suspected of having myocardial infarction is taken each time and dropped onto the fluorescence immunochromatographic test strip. Then, the test strip is immediately placed into the reader. The fluorescence spectrum data of the test strip is read once every 30 seconds. The reading is continued until the reaction is completed (e.g., when the fluorescence intensity signals at two adjacent time steps are substantially unchanged), thereby obtaining the fluorescence spectrum data of the entire reaction stage.

Step S2, constructing a signal preprocessing model to distinguish and label a reaction stage of the fluorescence spectrum data to obtain labeled fluorescence spectrum data, wherein the reaction stage includes reaction not started, reaction in progress, and reaction completed.

Distinguishing and labeling the reaction stage refers to labeling the fluorescence spectrum data of the entire reaction stage according to different reaction stages, to determine the fluorescence spectrum data respectively corresponding to the different reaction stages.

In some embodiments, the labeled fluorescence spectrum data may include a reaction-not-started signal, a reaction-in-progress signal, and a reaction-complete signal. The reaction-not-started signal refers to fluorescence spectrum data read before the sample to be tested migrates to the detection line of the fluorescence immunochromatographic test strip. The reaction-complete signal refers to fluorescence spectrum data read when a change rate of the fluorescence spectral signal is lower than a change rate threshold within a preset time period. For example, the change rate of the fluorescence spectral signal may be represented by a T/C line peak area ratio, and the reaction-complete signal may be fluorescence spectrum data read when the change rate of the T/C line peak area ratio within 30 seconds is less than 10%. The reaction-in-progress signal refers to fluorescence spectrum data read within a time period between the reaction not started and the reaction completed. The T/C line peak area ratio refers to a ratio of a T-line peak area to a C-line peak area. The change rate threshold may be set according to experience or actual needs.

In some embodiments, before constructing the signal preprocessing model, the processor may establish a reaction stage segmentation standard based on reaction kinetic features of the fluorescence immunochromatographic test strip, and distinguish and label the reaction stage of the fluorescence spectrum data based on the reaction stage segmentation standard.

The reaction kinetic features refer to data used for characterizing the rules and dynamic process of the fluorescence intensity signal changing over time. For example, the reaction kinetic features may include change curves over time of curve features (such as peak height, peak area, etc.) corresponding to the fluorescence spectrum data at a plurality of time points, or the like.

In some embodiments, the processor may perform curve fitting based on the curve features (such as the peak height, the peak area, etc.) corresponding to the fluorescence spectrum data at the plurality of time points to obtain the reaction kinetic features.

The reaction stage segmentation standard refers to a determination rule for dividing the fluorescence spectrum data into different reaction stages. In some embodiments, the segmentation standard corresponding to the reaction not started may include: a liquid sample flowing from a sample loading region of the test strip towards an absorbent pad, but has not yet reached the detection line (the T-line) and the control line (the C-line), indicated by no obvious feature peaks on the T-line and the C-line.

In some embodiments, the segmentation standard corresponding to the reaction in progress may include: the analyte (such as cTnI) in the liquid sample specifically binding with a pre-embedded fluorescence-labeled antibody on the test strip to form an “antigen-antibody-fluorescent label” complex and depositing on the detection line (the T-line), simultaneously, an excess fluorescence-labeled antibody continuing to flow and binding with the control line (the C-line), which leads to the fluorescence intensity signals of the T-line and the C-line gradually increasing over time, indicated by the feature peaks of the T-line and the C-line becoming increasingly obvious over time. The feature peaks becoming increasingly obvious may mean that a difference between a peak height and a curve platform height is larger compared to a previous time point.

In some embodiments, the segmentation standard corresponding to the reaction completed may include: the fluorescence intensity signals of the T-line and the C-line reaching a stable state, such as the change rate of the T/C line peak area ratio within a continuous 30 seconds being less than the change rate threshold (e.g., 10%).

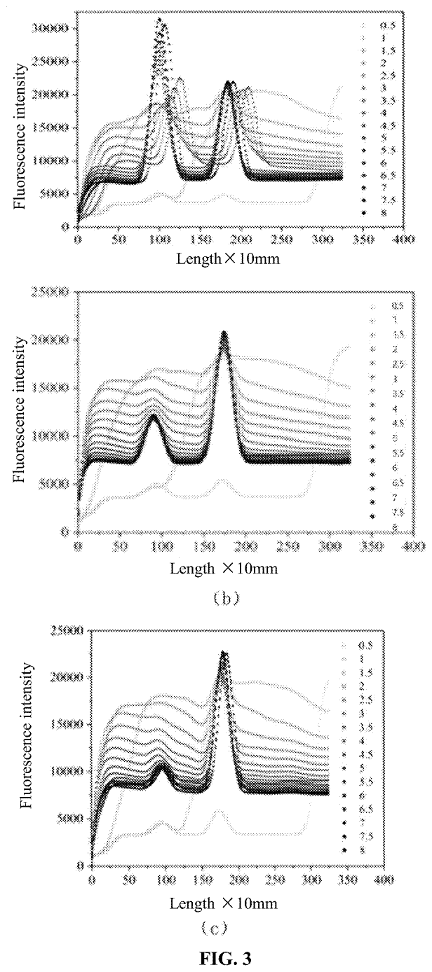

FIG. 3 is a schematic diagram illustrating typical fluorescence signal spectrums of a fluorescence immunochromatographic method according to some embodiments of the present disclosure. As shown in FIG. 3, (a) of FIG. 3 is a fluorescence intensity change diagram of the fluorescence immunochromatographic method for a positive sample, (b) of FIG. 3 is a fluorescence intensity change diagram of the fluorescence immunochromatographic method for a weak positive sample, and (c) of FIG. 3 is a fluorescence intensity change diagram of the fluorescence immunochromatographic method for a negative sample. The X-axes of (a), (b), and (c) all represent a length of the test strip, and the Y-axes represent a fluorescence intensity.

The negative sample refers to a sample to be tested that does not contain an analyte or whose analyte concentration is lower than a detection threshold. The positive sample refers to a sample to be tested that contains an analyte and whose analyte concentration is higher than the detection threshold. The weak positive sample refers to a sample to be tested that contains an analyte and whose analyte concentration is equal to or slightly higher than the detection threshold. The analyte concentration being slightly higher than the detection threshold may mean that a difference between the analyte concentration and the detection threshold is less than a difference threshold. The difference threshold and the detection threshold are preset based on actual needs.

As shown by the fluorescence spectrum data in (b), within a time period of 0 to 1 minute, the blood sample has not yet migrated to the T-line, while at 8 minutes, the reaction reaches completion. Therefore, the processor may label the fluorescence intensity signal read between 0-1.5 minutes as the reaction-not-started signal, label the fluorescence intensity signal read between 1.5-7.5 minutes as the reaction-in-progress signal, and label the fluorescence intensity signal read at 8 min as the reaction-complete signal.

In some embodiments, the processor may construct a two-layer fully connected neural network as the signal preprocessing model based on the established reaction stage segmentation standard.

The signal preprocessing model is configured for signal classification to distinguish and label the reaction stage for each piece of the fluorescence spectrum data, and to obtain the labeled fluorescence spectrum data.

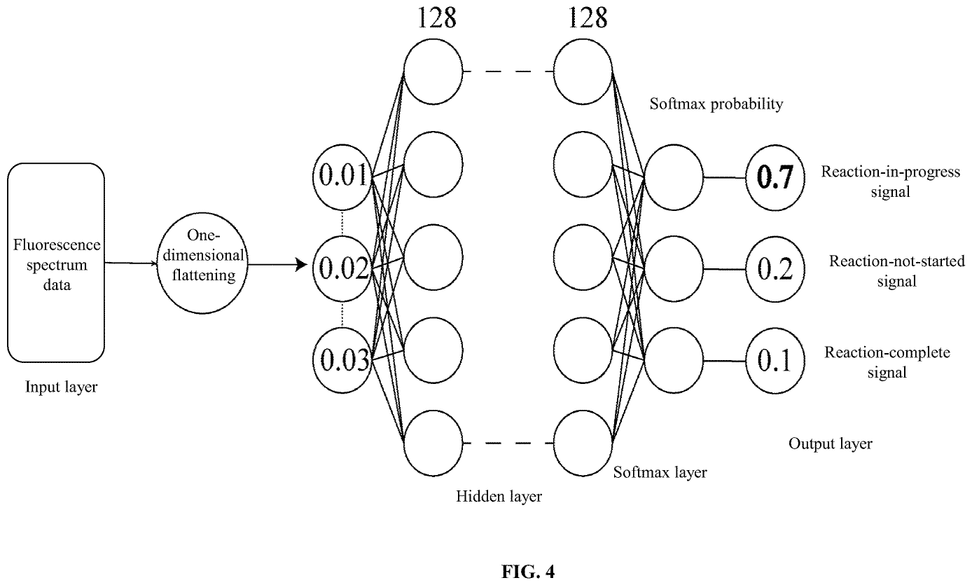

FIG. 4 is a schematic diagram illustrating a network structure of a signal preprocessing model according to some embodiments of the present disclosure.

In some embodiments, the network structure of the signal preprocessing model is as shown in FIG. 4, and the signal preprocessing model may include an input layer, a one-dimensional flattening layer, a hidden layer, a Softmax layer, and an output layer. The input layer may be configured for data processing on input fluorescence spectrum data. The one-dimensional flattening layer may be configured to erase position information in the fluorescence spectrum data and arrange fluorescence intensity signals in positional order to obtain a fluorescence intensity one-dimensional vector. The hidden layer may be configured to learn a mapping relationship between the fluorescence intensity one-dimensional vector and the reaction stage by adjusting weights and biases. The Softmax layer may be configured to convert a 128-dimensional vector output by the hidden layer into a probability distribution (i.e., Softmax probability). The output layer may be configured to distinguish and label the reaction stage for each piece of fluorescence spectrum data based on the probability distribution output by the Softmax layer, thereby obtaining the labeled fluorescence spectrum data.

Illustratively, for one piece of fluorescence spectrum data input into the signal preprocessing model, the probability distribution output by the Softmax layer is (0.7 (corresponding to the reaction-in-progress signal), 0.2 (corresponding to the reaction-not-started signal), 0.1 (corresponding to the reaction-complete signal)), then the piece of fluorescence spectrum data is labeled as the reaction-in-progress signal.

In some embodiments, a first training sample for training the signal preprocessing model includes sample fluorescence spectrum data, and the first training sample carries a first label. The first label includes an actual reaction stage of the sample fluorescence spectrum data.

In some embodiments, the signal preprocessing model may be trained by: inputting a plurality of first training samples with a first label into an initial signal preprocessing model; constructing a loss function through the first label and a prediction result of the initial signal preprocessing model; iteratively updating the initial signal preprocessing model based on the loss function; and completing the training of the signal preprocessing model when a loss function of the initial signal preprocessing model satisfies a preset ending condition. The preset ending condition may be convergence of the loss function, a count of iterations reaching a predetermined value, or the like.

Step S3, constructing a signal feature extraction model to extract a fluorescence intensity signal feature vector from the labeled fluorescence spectrum data.

The fluorescence intensity signal feature vector refers to a feature vector for characterizing the morphological features of the fluorescence intensity signal. The fluorescence intensity signal feature vector may also be referred to as a continuous time step feature vector. The morphological features of the fluorescence intensity signal may include a T-line peak area, a C-line peak area, a T-line fluorescence intensity, a C-line fluorescence intensity, a T/C line peak area ratio, a T/C line fluorescence intensity ratio, and a background fluorescence intensity. In some embodiments, extracting the fluorescence intensity signal feature vector may include: extracting a fluorescence intensity signal feature vector from the fluorescence spectrum data corresponding to the reaction not started; extracting a fluorescence intensity signal feature vector from the fluorescence spectrum data corresponding to the reaction in progress (referred to as a reaction-in-progress feature vector); and extracting a fluorescence intensity signal feature vector from the fluorescence spectrum data corresponding to the reaction completed (referred to as a reaction-complete feature vector).

In some embodiments, the reaction-complete feature vector may be a reaction-complete feature vector obtained by extraction using the signal feature extraction model, or a reaction-complete feature vector obtained by time series prediction using a time series prediction model. For descriptions of the time series prediction model, refer to Step S4.

In some embodiments, the processor may obtain multi-dimensional feature values of the fluorescence spectrum data corresponding to the reaction in progress, and train to obtain the signal feature extraction model by taking the fluorescence spectrum data corresponding to the reaction in progress as an input and the multi-dimensional feature values as an output.

The multi-dimensional feature values refer to feature indicators for describing the morphological features of the fluorescence spectrum data. In some embodiments, the multi-dimensional feature values include, but are not limited to, the T-line peak area, the C-line peak area, the T-line fluorescence intensity, the C-line fluorescence intensity, the T/C line peak area ratio, the T/C line fluorescence intensity ratio, and the background fluorescence intensity.

The T-line fluorescence intensity may be a maximum value of a fluorescence intensity signal detected within a T-line region. The C-line fluorescence intensity may be a maximum value of a fluorescence intensity signal detected within a C-line region.

The T/C line fluorescence intensity ratio refers to a ratio of the T-line fluorescence intensity to the C-line fluorescence intensity.

The background fluorescence intensity refers to an average fluorescence intensity signal read in a non-reaction region of the fluorescence immunochromatographic test strip (e.g., a blank region between the T-line and the C-line).

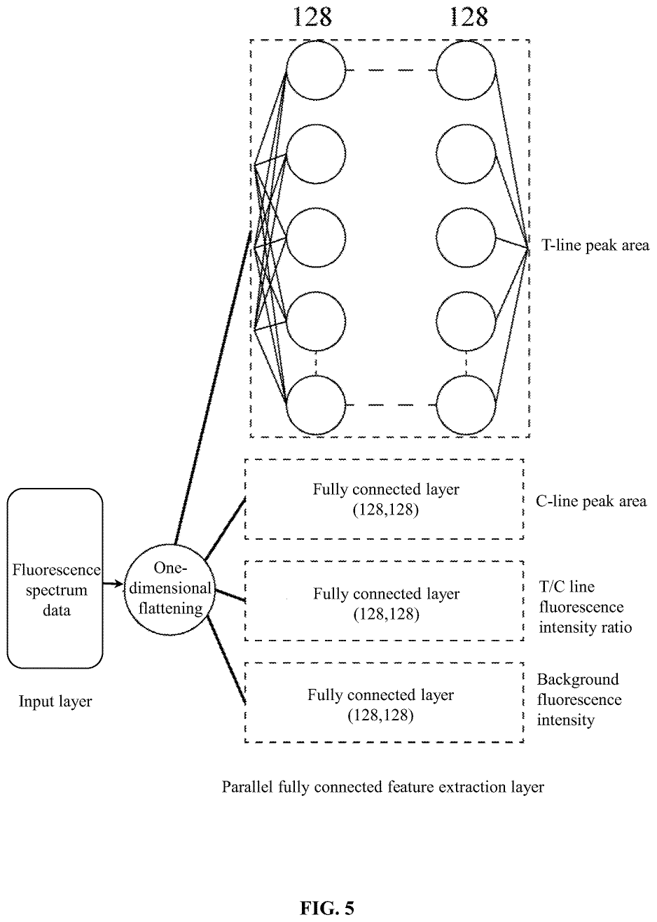

FIG. 5 is a schematic diagram illustrating a network structure of a signal feature extraction model according to some embodiments of the present disclosure.

In some embodiments, the network structure of the signal feature extraction model is as shown in FIG. 5, and the signal feature extraction model may include an input layer, a one-dimensional flattening layer, a parallel fully connected feature extraction layer, and an output layer. The input layer is configured to input fluorescence spectrum data corresponding to the reaction in progress. The one-dimensional flattening layer is configured to flatten two-dimensional fluorescence spectrum data into a one-dimensional vector (by arranging fluorescence intensity signals in positional order to obtain the one-dimensional vector). The parallel fully connected feature extraction layer includes four independent fully connected layers, which are a first branch fully connected layer, a second branch fully connected layer, a third branch fully connected layer, and a fourth branch fully connected layer. An output feature of the first branch fully connected layer is the T-line peak area, and the T-line peak area may quantify the total fluorescence signal of the detection line (T-line) and directly reflect the analyte concentration. An output feature of the second branch fully connected layer is the C-line peak area, and the C-line peak area may quantify the signal intensity of the control line (C-line) and verify the validity of the test strip. An output feature of the third branch fully connected layer is the T/C line fluorescence intensity ratio. An output feature of the fourth branch fully connected layer is the background fluorescence intensity, and the T/C line fluorescence intensity ratio and the background fluorescence intensity may be used for standardizing a signal (eliminating environmental interference) and background noise. The input dimension and the output dimension of each branch fully connected layer are both 128 dimensions (as indicated by “128” in FIG. 5). The output of the output layer may include the T-line peak area, the C-line peak area, the T/C line fluorescence intensity ratio, and the background fluorescence intensity.

In some embodiments, a second training sample for training the signal feature extraction model includes fluorescence spectrum data of a sample reaction-in-progress stage, and the second training sample has a second label. The second label includes actual multi-dimensional feature values of the second training sample. The training process of the signal feature extraction model is similar to the training process of the signal preprocessing model.

Step S4, constructing the time series prediction model to perform time series prediction processing on the reaction-in-progress feature vector to obtain the reaction-complete feature vector.

In some embodiments, when constructing the time series prediction model, the processor may combine a plurality of the fluorescence intensity signal feature vectors into a feature vector, set a time window to obtain a feature vector sequence of a fixed length, slide the time window to capture a plurality of feature vector sequences covering an entire reaction stage (also referred to as an entire reaction-in-progress stage), obtain training samples, and input the training samples into a Transformer model to construct the time series prediction model.

As shown in FIG. 5, a single vertical column (for example, the vertical column corresponding to t1) represents a fluorescence intensity signal feature vector (including a reaction-in-progress feature vector and a reaction-complete feature vector), and the processor may arrange a plurality of fluorescence intensity signal feature vectors in chronological order to obtain a combined feature vector.

The time window (also referred to as a sliding window) refers to a time interval of a fixed length. As shown in FIG. 5, the length of the time window may be 4, which means covering a duration of two minutes. The feature vector sequence of a fixed length refers to a sequence composed of the reaction-in-progress feature vectors truncated according to the time window.

In some embodiments, the processor may further adjust the length of the time window based on a complexity of the fluorescence spectrum data and an identification condition.

The complexity refers to a quantified score for evaluating the degree of complexity of the fluorescence spectrum data.

In some embodiments, the processor may determine the complexity of the fluorescence spectrum data based on data features of the fluorescence spectrum data. The fluorescence spectrum data at each time point corresponds to a complexity.

The data features of the fluorescence spectrum data refer to data related to the intrinsic attributes of the fluorescence spectrum data. In some embodiments, the data features include a signal-to-noise ratio, a chromatographic flow rate, and a waveform integrity. The chromatographic flow rate refers to a rate at which the liquid sample moves on the test strip. The waveform integrity may be represented by a skewness of a T-line waveform.

In some embodiments, the processor may directly obtain the signal-to-noise ratio and the chromatographic flow rate through the reader. The processor may further obtain the waveform integrity by performing a statistical determination on the fluorescence spectrum data (for example, determining a third-order time point (skewness)).

In some embodiments, the processor may normalize the data features of the fluorescence spectrum data. After the normalization, the processor obtains the complexity of the fluorescence spectrum data by performing a weighted summation on the normalized data.

For example, the processor may obtain the complexity of the fluorescence spectrum data through the following formula (1):

K=k₁×a1+k₂×|a₂−m|+k₃×|a3| (1)
where K represents the complexity of the fluorescence spectrum data; k₁, k₂, and k₂ are all preset weights, and k₁ is a negative weight (the lower the signal-to-noise ratio, the greater the background interference, and the higher the complexity); k₂ and k₃ are positive weights; a1 represents the signal-to-noise ratio; a2 represents the chromatographic flow rate; a3 represents the waveform integrity; and m represents a standard chromatographic flow rate. The standard chromatographic flow rate may be preset by experience or actual needs.

In some embodiments, when determining the complexity using the above formula (1), the processor may also perform the weighted summation by adding physiological parameters of a user (such as a hematocrit value of a patient) with preset weights. The physiological parameters are obtained through pre-input.

The identification condition refers to a determination condition for determining whether the reaction-complete feature vector needs to be re-predicted. In some embodiments, the identification condition may include that the average complexity of the fluorescence spectrum data at a plurality of time points is less than a complexity threshold. The complexity threshold is preset based on historical experience.

In some embodiments, the processor may query a preset length table to determine the length of the time window based on a difference between the average complexity and the complexity threshold in the identification condition. The preset length table is preset based on historical experience, and includes a plurality of values and the length of the time window corresponding to each value. The greater the difference between the average complexity and the complexity threshold, the longer the length of the time window.

In some embodiments of the present disclosure, the length of the time window is dynamically and adaptively adjusted based on the average complexity of the fluorescence spectrum data, which ensures the detection accuracy of complex samples (such as high-viscosity blood samples) while also optimizing the overall detection efficiency.

In some embodiments, the processor slides the time window, obtains a feature vector sequence with each slide until the time window covers the last reaction-in-progress feature vector, thereby capturing a plurality of feature vector sequences covering the entire reaction stage. Each feature vector sequence serves as a third training sample. The third training sample carries a third label. The third label includes an actual reaction-complete feature vector corresponding to the third training sample.

In some embodiments, the processor inputs the training samples (the third training samples) into the Transformer model and adjust the hyperparameters of the Transformer model to construct the time series prediction model.

In some embodiments, the training process of the Transformer model is similar to the training process of the signal preprocessing model.

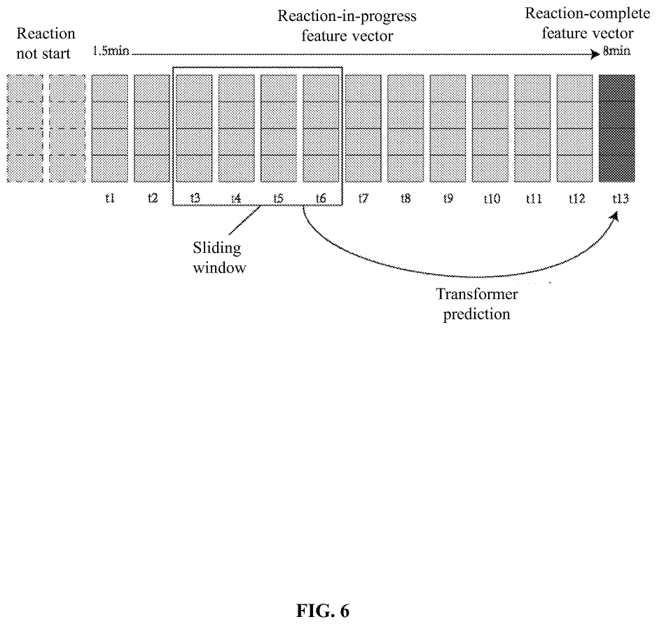

FIG. 6 is a schematic diagram illustrating a network structure of a time series prediction model according to some embodiments of the present disclosure. In some embodiments, the network structure of the time series prediction model is as shown in FIG. 6.

For example, constructing the time series prediction model may include taking fluorescence intensity signal feature vectors of 13 time steps obtained on a single test strip as a group (wherein a plurality of groups of feature vectors are obtained through a plurality of test strips). The time window is set to 4. The time window slides from a first time step (t1) until a 12-th time step (t12), as shown in FIG. 6.

The processor performs this operation on each group of feature vectors and obtains a large amount of training data. The processor calls the Transformer model from a deep learning library and defines the network structure, such as the input dimension of 4, the hidden layer dimension of 128, the count of attention heads of 2, and the count of encoder layers of 2. Absolute positional encoding is adopted to enable the Transformer model to perceive the position of time steps in the input training sample sequence. The processor extracts the hidden state of the last position of the input training sample sequence as the representation of the entire sequence, and generates a final prediction result through the hidden state. Finally, the processor takes the time window as an input and the reaction-complete feature vector of t13 as an output, and trains the Transformer model for 1000 iterations, and the mean square error is 0.28.

In some embodiments of the present disclosure, according to a multi-feature vector combination (such as the T/C line peak area ratio, the fluorescence intensity, etc.), the reaction dynamics can be fully captured, thereby avoiding single feature deviations. A sliding time window mechanism may convert continuously sampled time series data into trainable sequence samples, which not only expands the effective data amount but also retains the time dependency of reaction kinetics. Through hyperparameter tuning, the time series prediction model is enabled to converge quickly and achieve high prediction accuracy (where the mean square error is 0.28 in the embodiment). Through multi-feature fusion, time series modeling, and parameter optimization, the Transformer model is enabled to accurately predict an endpoint signal based on early reaction data.

In some embodiments, the time series prediction model may also include a dual-channel embedding layer and a standard Transformer structure that are sequentially connected. In some embodiments, the processor may also determine an ideal reaction curve based on test strip parameters and determine an actual reaction curve based on the fluorescence spectrum data. The processor determines a residual sequence based on the ideal reaction curve and the actual reaction curve, and inputs the residual sequence and the ideal reaction curve into the dual-channel embedding layer to obtain a residual vector sequence and an ideal feature sequence. The processor may input the residual vector sequence and the ideal feature sequence into the standard Transformer structure to predict the reaction-complete feature vector.

The dual-channel embedding layer is configured to convert low-dimensional data (such as the residual sequence and the ideal reaction curve) into high-dimensional embedding vectors, so that the standard Transformer structure can process the high-dimensional embedding vectors. In some embodiments, the dual-channel embedding layer may include two independent and parallel fully connected layers, which are used for processing the ideal reaction curve and the residual sequence, respectively.

The standard Transformer structure may include the Transformer model described above, or the like.

The test strip parameters refer to parameters that describe the physical properties and biochemical properties of the test strip. In some embodiments, the test strip parameters include a pore size, a chromatographic distance, an association rate constant, a dissociation rate constant, or the like. The test strip parameters may be determined by obtaining an input from technical personnel.

The ideal reaction curve refers to a curve obtained under ideal conditions (such as no noise, etc.), which represents the change of the data features of the fluorescence spectrum data over time.

The actual reaction curve refers to a curve that characterizes the data features over time of the actual fluorescence spectrum data.

In some embodiments, the ordinate of the ideal reaction curve and the actual reaction curve may be the T/C line peak area ratio and the T-line peak area, and the abscissa may be the time point.

In some embodiments, the processor may determine a time and an instantaneous flow rate of the liquid sample flowing through the T-line based on the pore size and the chromatographic distance through a chromatographic flow rate equation (such as the Washburn equation), etc. The processor may combine an antigen-antibody binding kinetic equation (such as the Langmuir adsorption kinetic equation) to construct a set of equations. The processor iteratively solves the set of equations through a numerical integration algorithm (such as the fourth-order Runge-Kutta algorithm), obtains T/C line peak area ratios and T-line peak areas at a plurality of time points, and constructs a theoretical reaction curve.

In some embodiments, the processor may utilize a fitting algorithm (such as the Least Squares), etc., to perform curve fitting on the T/C line peak area ratios and the T-line peak areas of the fluorescence spectrum data at the plurality of time points to obtain the actual reaction curve.

The residual sequence refers to a sequence composed of residuals at the plurality of time points. The residual may be a difference between the ordinate of the ideal reaction curve and the ordinate of the actual reaction curve at the same abscissa. The residual includes a residual of the T/C line peak area ratio and a residual of the T-line peak area.

The residual vector sequence and the ideal feature sequence refer to high-dimensional vector sequences obtained after the residual sequence and the ideal reaction curve are respectively processed by the dual-channel embedding layer.

For example, taking the full connection layer for processing the residual sequence as an example, the process of the processor converting the residual sequence into the residual vector sequence via the full connection layer may include the following. For the residual at each time point in the residual sequence, the processor may perform a matrix operation on the residual and perform a nonlinear transformation via an activation function to obtain a residual vector corresponding to the time point. Illustratively, formula (2) may be expressed as:

$${E}_{r e s}^{( t )} = R e L U ( [ s 1 , s 2 ] \times {W}_{r e s} + {B}_{r e s} ) \qquad ( 2 )$$

where

$${E}_{res}^{( t )}$$

represents a residual vector at a time point t, ReLU represents the activation function (such as a Gaussian error linear unit); s1 represents a residual of the T/C line peak area ratio at the time point t, s2 represents a residual of the T-line peak area at the time point t, Wᵣₑₛ represents a preset weight matrix, and Bᵣₑₛ represents a preset bias vector.

In some embodiments of the present disclosure, by introducing the ideal reaction curve and the residual sequence, which are constrained by physical and biochemical kinetic equations, into a pure data-driven Transformer model, prediction robustness and interpretability of the model under extreme environments or abnormal biochemical reactions are effectively improved.

In some embodiments, in response to determining that the complexity satisfies the identification condition, the processor predicts the reaction-complete feature vector based on the reaction-in-progress feature vector through the time series prediction model. In response to determining that the complexity does not satisfy the identification condition, the processor determines a morphological feature vector based on the fluorescence spectrum data corresponding to the reaction in progress, and predicts the reaction-complete feature vector based on the morphological feature vector and the reaction-in-progress feature vector by the time series prediction model. For descriptions of the identification condition, reference may be made to the above and related descriptions thereof. For descriptions of the complexity, the reaction-in-progress feature vector, the time series prediction model, and the reaction-complete feature vector, reference may be made to the above and related descriptions thereof.

The morphological feature vector refers to a vector extracted from the fluorescence spectrum data and used for describing the morphological features of the T-line and the C-line. In some embodiments, elements of the morphological feature vector include a skewness, a kurtosis, a full width at half maximum (FWHM), and a peak splitting condition. The kurtosis is used for describing a sharpness or a flatness of a waveform. The full width at half maximum (FWHM) refers to a width at half of a peak height, and reflects a degree of diffusion of a band. The peak splitting condition may reflect whether double peaks or other such phenomena appear in the T-line or the C-line.

In some embodiments, the processor may perform statistical analysis on a local spectrum interval where the T-line and the C-line are located, determine the skewness and the kurtosis, and measure an intercept width at a 50% position of a peak maximum to obtain the full width at half maximum. The processor may further employ a spectral peak-finding algorithm (such as a second derivative approach) to detect whether two or more maximum points exist in the local interval, and if such points exist, the condition is marked as peak splitting, and the split interval or the secondary peak height is used as a feature value.

In some embodiments, the processor may concatenate the morphological feature vector and the reaction-in-progress feature vector to form an augmented feature vector with a higher dimension, and input the augmented feature vector to the time series prediction model to predict the reaction-complete feature vector through the time series prediction model. Since the input dimension increases, the time series prediction model adjusts a size of the weight matrix accordingly, thereby simultaneously learning a kinetic trend of the reaction (via the reaction-in-progress feature vector) and waveform evolution details (via the morphological feature vector).

In some embodiments of the present disclosure, by dynamically triggering extraction and fusion of two-dimensional enhanced morphological features based on the complexity, data morphological evolution information during the reaction process is effectively preserved, which avoids high-concentration false negatives or misdiagnosis caused by feature loss under abnormal biochemical reactions or complex samples.

Step S5, constructing a detection result output model to perform diagnosis based on the reaction-complete feature vector to obtain the detection result of the analyte in the sample to be tested.

The detection result of the analyte refers to a determination result of the concentration of the analyte (such as cTnI) in the sample to be tested. In some embodiments, the detection result of the analyte may include “negative”, “weakly positive”, and “positive”. For descriptions of “negative”, “weakly positive”, and “positive”, reference may be made to Step S2.

In some embodiments, the processor constructing the detection result output model includes: setting a plurality of analyte concentration thresholds corresponding to a plurality of detection results of the analyte in the sample to be tested; obtaining the reaction-complete feature vector of the analyte; detecting the reaction-complete feature vector of the analyte based on the plurality of analyte concentration thresholds to obtain the detection result of the analyte of the sample to be tested; and then training the detection result output model based on the detection result of the analyte and the reaction-complete feature vector to obtain a constructed detection result output model.

The analyte concentration threshold refers to a critical concentration value used for distinguishing different detection results. In some embodiments, the processor may inversely determine the analyte concentration through the T/C line peak area ratio at the reaction completed, and set a blood sample with an analyte concentration less than a first threshold (such as 0.1 ng/mL) as negative, a blood sample with an analyte concentration not less than the first threshold (such as 0.1 ng/mL) but not greater than a second threshold (such as 0.3 ng/mL) as weakly positive, and a blood sample with an analyte concentration greater than the second threshold as positive.

In some embodiments, the processor may use the detection result of the analyte as a label, and use the reaction-complete feature vector as an input to train and obtain the detection result output model.

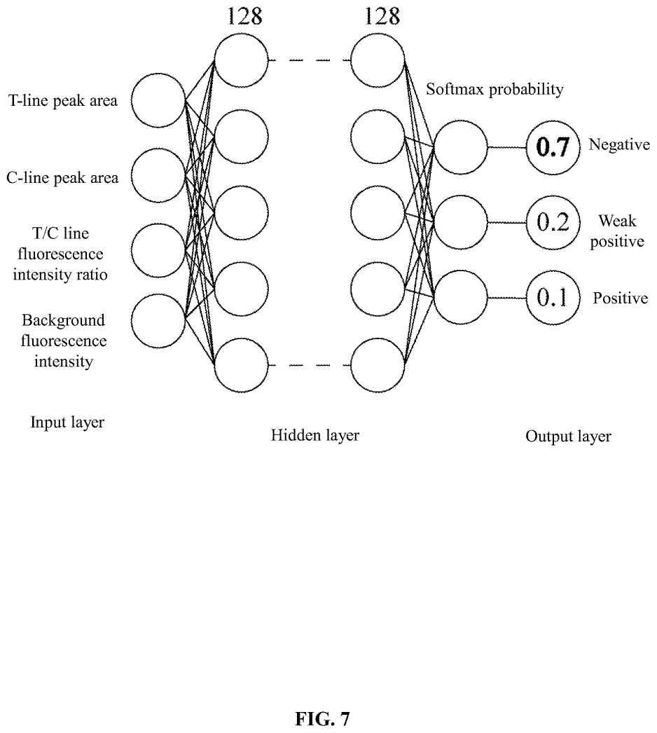

FIG. 7 is a schematic diagram illustrating a network structure of a detection result output model according to some embodiments of the present disclosure.

In some embodiments, the network structure of the detection result output model is as shown in FIG. 7, and the detection result output model includes an input layer, a hidden layer, and an output layer. The input layer is configured to input the reaction-complete feature vector. A first layer of the hidden layer maps 4-dimensional original features to a 128-dimensional space to extract nonlinear combination features. A second layer is configured to extract deep abstract features from the extracted nonlinear combination features to capture an interaction relationship between the T/C line fluorescence intensity ratio and the background fluorescence intensity. The output layer is configured to determine the analyte concentration based on the interaction relationship between the T/C line fluorescence intensity ratio and the background fluorescence intensity, and then output the detection result of the analyte and the analyte concentration.

Illustratively, when the analyte concentration is lower than the first threshold, the processor determines the sample to be tested as negative. When the analyte concentration is between the first threshold and the second threshold, the processor determines the sample to be tested as weakly positive. When the analyte concentration is higher than the second threshold, the processor determines the sample to be tested as positive.

Step S6, inputting collected fluorescence intensity signals of the reaction in progress into the signal preprocessing model for time-domain differentiation labeling, extracting the fluorescence intensity signal feature vector through the signal feature extraction model, predicting the reaction-complete feature vector through the time series prediction model, and obtaining the detection result of the analyte in the sample to be tested through the detection result output model.

The fluorescence intensity signal of the reaction in progress refers to a fluorescence intensity signal collected during the entire reaction stage, namely, the aforementioned fluorescence spectrum data.

In some embodiments, the processor may integrate the trained models (the signal preprocessing model, the signal feature extraction model, the time series prediction model, and the detection result output model) to construct a model structure as shown in FIG. 2. The processor sequentially inputs time-continuous fluorescence spectrum data to the signal preprocessing model, removes signals that cannot or do not need to be predicted (such as the reaction-not-started signal), to obtain the reaction-in-progress signal. The processor converts the reaction-in-progress signal into the reaction-in-progress feature vector composed of four features through the signal feature extraction model. The processor inputs the reaction-in-progress feature vector of four consecutive time steps to the time series prediction model to obtain the reaction-complete feature vector (such as the aforementioned t13 feature vector). The processor transmits the reaction-complete feature vector to the detection result output model to obtain the analyte concentration and the detection result of the analyte in the sample to be tested. Through the above operations, a traditional 15-minute detection process can be compressed to 3.5 minutes, while maintaining a diagnosis accuracy rate of 95% or more, which provides golden treatment time for critical diseases such as AMI or the like.

In some embodiments of the present disclosure, through deep learning, predictive detection analysis is performed on the analyte in the sample to be tested based on the reaction-in-progress signal. This enables predictive detection of the analyte without waiting for the analyte to react completely on the fluorescence immunochromatographic test strip, thereby satisfying a rapid detection requirement for the analyte by a fluorescence immunochromatography approach.

In some embodiments, the processor may further generate an electrocardiogram monitoring instruction based on the analyte concentration and send the electrocardiogram monitoring instruction to an electrocardiogram monitor, and control a recording frequency of the electrocardiogram monitor based on the electrocardiogram monitoring instruction.

The electrocardiogram monitoring instruction refers to an instruction used for controlling operation of the electrocardiogram monitor. In some embodiments, the electrocardiogram monitoring instruction may include the recording frequency of the electrocardiogram monitor or the like. The processor may query a preset frequency table based on the analyte concentration to determine the recording frequency corresponding to the analyte concentration, and then generate the electrocardiogram monitoring instruction. The preset frequency table is preset based on historical data, and includes a plurality of correspondence relationships between analyte concentration intervals and recording frequencies. The higher the analyte concentration, the higher the recording frequency.

In some embodiments, the processor may send the electrocardiogram monitoring instruction to a main control chip of the electrocardiogram monitor (such as a microcontroller unit or a FPGA). After receiving the electrocardiogram monitoring instruction, the main control chip decodes the electrocardiogram monitoring instruction and extracts the recording frequency. The main control chip writes a new recording frequency into a control register of the electrocardiogram monitor through an internal bus. The main control chip, according to the recording frequency written into the control register, changes an interrupt period of a timer that controls an analog-to-digital converter (ADC) for sampling, or adjusts a frequency division ratio of a system clock generator.

For example, the timer sends a sampling trigger level to the ADC every 4 milliseconds (i.e., 250 Hz). After the main control chip receives the new recording frequency, the timer is adjusted to send the sampling trigger level to the ADC every 1 millisecond (i.e., 1000 Hz), thereby directly improving the sampling and recording frequency of an electrocardiogram waveform at a hardware physical layer level.

In some embodiments of the present disclosure, automated linkage between the detection result and physiological monitoring devices is achieved by generating and sending control instructions, which provides more timely and high-frequency auxiliary physiological vital sign data for medical personnel targeting critical conditions (such as myocardial infarction).

In some embodiments, if the analyte concentration satisfies a concentration condition, the processor generates an infusion pump control instruction based on the analyte concentration and the physiological parameters of the user (such as a patient), sends the infusion pump control instruction to a controller of an infusion pump, and controls a pumping rate of the infusion pump based on the infusion pump control instruction.

The concentration condition refers to a condition used to determine whether to adjust the pumping rate of the infusion pump. In some embodiments, the concentration condition may include that the analyte concentration is greater than a safety concentration threshold. The safety concentration threshold may be preset based on historical experience.

The physiological parameters refer to indicators reflecting vital signs of the patient. In some embodiments, the physiological parameters include the weight of the patient, the age of the patient, the type of disease, or the like. The physiological parameters may be obtained via input.

The infusion pump control instruction refers to an instruction used to control the operation of the infusion pump. In some embodiments, the infusion pump control instruction may include the pumping rate of the infusion pump or the like.

In some embodiments, the processor may construct a matching feature vector based on the analyte concentration and the physiological parameters of the patient, query a vector database for a reference feature vector satisfying a matching condition with the matching feature vector, and use a label corresponding to the reference feature vector as the pumping rate of the infusion pump. The matching condition includes a maximum similarity between vectors. The vector similarity is negatively correlated with a vector distance. The vector distance includes a Euclidean distance or the like.

In some embodiments, the vector database may be constructed based on historical data, and includes a plurality of reference feature vectors and corresponding labels. For example, the processor may screen historical treatment processes from the historical data that satisfy a preset condition, construct reference feature vectors based on historical analyte concentrations and historical physiological parameters in the historical treatment processes, and use historical pumping rates used in the historical treatment processes as labels.

In some embodiments, the preset condition may be that after the infusion pump infuses the patient at the historical pumping rate, the treatment effect on the patient is good (e.g., ST segment regression of the electrocardiogram of the patient, reduction of myocardial infarction area, or relief of chest pain symptoms of the patient).

In some embodiments, the processor may send the infusion pump control instruction to a control unit of the infusion pump. In response to receiving the infusion pump control instruction, the control unit of the infusion pump adjusts the pumping rate of the medicine according to the infusion pump control instruction.

In some embodiments of the present disclosure, by pre-adjusting the pumping parameters of the infusion pump, the manual setup time of equipment by medical personnel during an emergency rescue process is shortened.

In some embodiments, if the analyte concentration satisfies the concentration condition, the processor may also generate an unlocking instruction based on the analyte concentration, the physiological parameters of the user, and medicine cabinet information, send the unlocking instruction to a smart medicine cabinet, control the smart medicine cabinet to unlock a target drawer based on the unlocking instruction, and trigger a prompting device of the target drawer.

The medicine cabinet information refers to data describing the type of medicine stored in the smart medicine cabinet. In some embodiments, the medicine cabinet information includes the type of medicine stored in each drawer in the smart medicine cabinet. The processor may perform real-time data synchronization with a control system of the smart medicine cabinet to obtain the medicine cabinet information.

The unlocking instruction refers to an instruction used to instruct the smart medicine cabinet to open a specific drawer. In some embodiments, the unlocking instruction includes identification information (such as a drawer number or the like) of the drawer to be unlocked (i.e., the target drawer).

In some embodiments, the processor determines the target drawer through a clustering analysis algorithm based on the analyte concentration, the physiological parameters, and the medicine cabinet information. Illustratively, the processor may construct a target vector based on the analyte concentration, the physiological parameters, and the medicine cabinet information, and construct clustering vectors based on a plurality of sets of historical analyte concentrations, historical physiological parameters, and historical medicine cabinet information. The label of the clustering vector may be the medicine actually used in the historical treatment process corresponding to the clustering vector.

In some embodiments, the processor may perform clustering on a plurality of clustering vectors and the target vector to obtain a plurality of clustering clusters, designate the clustering cluster containing the target vector as a target cluster, and determine a label with the highest frequency in the target cluster as a medicine to be used (designated as a target medicine). The processor determines the target drawer and generates the unlocking instruction according to the target medicine and the type of medicine stored in each drawer in the medicine cabinet information, and sends the unlocking instruction to the smart medicine cabinet.

In some embodiments, after receiving the unlocking instruction, a main control chip of the smart medicine cabinet drives an electronic lock corresponding to the target drawer to unlock, and triggers the prompting device of the target drawer. The prompting device includes an indicator light, a buzzer, a display screen, or the like. When the target drawer is unlocked, the prompting device provides a prompt via a visual signal or an audible signal.

In some embodiments of the present disclosure, directly controlling the physical opening and prompting of the smart medicine cabinet through the detection result improves the accuracy and response speed of obtaining emergency rescue medicine in emergency scenarios.

One or more embodiments of the present disclosure also provide a device for rapid reading and analysis of a fluorescence immunochromatographic test strip based on deep learning for rapid in vitro diagnosis of cTnI (hereinafter referred to as the device). The device may be a device with a data processing function, such as a PC, a laptop, a server, an embedded device, etc., and has a communication interface, a memory, a processor, etc.

In some embodiments, the communication interface includes an I/O interface, a physical interface, and a logical interface for realizing component interconnection within the device, and an interface for realizing interconnection between the device and other apparatuses (e.g., other computing apparatuses or user apparatuses). The physical interface may be an Ethernet interface, a fiber optic interface, an Asynchronous Transfer Mode (ATM) interface, or the like. The user apparatuses may be a display, a keyboard, or the like.

In some embodiments, the memory may be various types of storage media, such as a random access memory (RAM), a read-only memory (ROM), a non-volatile RAM (NVRAM), a flash memory, optical storage, a hard disk, a programmable ROM (PROM), an erasable PROM (EPROM), an electrically erasable PROM (EEPROM), or the like.

In some embodiments, the processor may be a general-purpose processor, and the general-purpose processor may call a program for rapid reading and analysis of a fluorescence immunochromatographic test strip based on deep learning for rapid in vitro diagnosis of cTnI (hereinafter referred to as the program) stored in the memory, and perform the method for rapid reading and analysis of a fluorescence immunochromatographic test strip based on deep learning provided in embodiments of the present disclosure. For example, the general-purpose processor may be a central processing unit (CPU). The process executed when the program is called may refer to the various embodiments of the method for rapid reading and analysis of a fluorescence immunochromatographic test strip of the present disclosure, and is not repeatedly described herein.

One or more embodiments of the present disclosure also provide a non-transitory readable storage medium. The readable storage medium stores the program, wherein when the program is executed by the processor, the method for rapid reading and analysis of a fluorescence immunochromatographic test strip as described above is implemented.

The method implemented when the program is executed may refer to the various embodiments of the method for rapid reading and analysis of a fluorescence immunochromatographic test strip of the present disclosure, and is not repeatedly described herein.

It should be noted that the above-mentioned sequence numbers of the embodiments of the present disclosure are merely for description and do not represent the superiority or inferiority of the embodiments.

Through the description of the above embodiments, those skilled in the art can clearly understand that the manner of the above-mentioned embodiments can be implemented by means of software plus a necessary general-purpose hardware platform. Certainly, it can also be implemented by hardware, however, in many cases, the former is a better implementation manner. Based on such an understanding, the technical solution of the present disclosure, in essence or the part that contributes to the prior art, can be embodied in the form of a software product. The computer software product is stored in a storage medium as described above (such as ROM/RAM, a magnetic disk, an optical disc), and includes a plurality of instructions for enabling a terminal device to perform the manners described in various embodiments of the present disclosure.

The foregoing are merely preferred embodiments of the present disclosure and are not intended to limit the patent scope of the present disclosure thereby. Any equivalent structures or equivalent process transformations made by utilizing the contents of the present disclosure and the accompanying drawings, or directly or indirectly applied to other related technical fields, are similarly included within the patent protection scope of the present disclosure.

What is claimed is:

1. A method for rapid reading and analysis of a fluorescence immunochromatographic test strip based on deep learning, comprising:
obtaining fluorescence spectrum data of a sample to be tested on the fluorescence immunochromatographic test strip;
constructing a signal preprocessing model to distinguish and label a reaction stage of the fluorescence spectrum data to obtain labeled fluorescence spectrum data, wherein the reaction stage includes reaction not started, reaction in progress, and reaction completed;
constructing a signal feature extraction model to extract a fluorescence intensity signal feature vector from the labeled fluorescence spectrum data;
constructing a time series prediction model to perform time series prediction processing on a reaction-in-progress feature vector to obtain a reaction-complete feature vector;
wherein constructing the time series prediction model includes:
combining a plurality of fluorescence intensity signal feature vectors into a feature vector;
setting a time window to obtain a feature vector sequence of a fixed length;
sliding the time window to obtain a plurality of feature vector sequences covering an entire reaction stage, and obtaining training samples; and
inputting the training samples into a Transformer model to construct the time series prediction model;
constructing a detection result output model to perform a diagnosis based on the reaction-complete feature vector to obtain a detection result of an analyte in the sample to be tested; and
inputting collected fluorescence intensity signals of the reaction in progress into the signal preprocessing model for time-domain distinguishing and labeling, extracting the plurality of fluorescence intensity signal feature vectors through the signal feature extraction model, predicting the reaction-complete feature vector through the time series prediction model, and obtaining the detection result of the analyte in the sample to be tested through the detection result output model.

2. The method of claim 1, wherein the obtaining fluorescence spectrum data of a sample to be tested on the fluorescence immunochromatographic test strip includes:
dropping the sample to be tested onto the fluorescence immunochromatographic test strip, reading a fluorescence intensity signal at each position on the fluorescence immunochromatographic test strip at a preset frequency until a reaction is completed, to obtain the fluorescence spectrum data.

3. The method of claim 1, wherein a reaction-not-started signal is a fluorescence spectral signal read before the sample to be tested migrates to a detection line of the fluorescence immunochromatographic test strip; a reaction-complete signal is a fluorescence spectral signal read when a change rate of the fluorescence spectral signal is lower than a change rate threshold within a preset time period; and a reaction-in-progress signal is a fluorescence spectral signal read within a time period between the reaction not started and the reaction completed.

4. The method of claim 1, wherein the constructing a signal preprocessing model includes:
establishing a reaction stage segmentation standard based on reaction kinetic features of the fluorescence immunochromatographic test strip; and
distinguishing and labeling the reaction stage of the fluorescence spectrum data based on the reaction stage segmentation standard.

5. The method of claim 1, wherein the constructing a signal feature extraction model includes:
obtaining multi-dimensional feature values of the fluorescence spectrum data corresponding to the reaction in progress; and
training to obtain the signal feature extraction model by using the fluorescence spectrum data corresponding to the reaction in progress as an input and the multi-dimensional feature values as an output.

6. The method of claim 5, wherein the multi-dimensional feature values include a T-line peak area, a C-line peak area, a T-line fluorescence intensity, a C-line fluorescence intensity, a T/C line peak area ratio, a T/C line fluorescence intensity ratio, and a background fluorescence intensity.

7. The method of claim 1, wherein the constructing a detection result output model includes:
setting a plurality of analyte concentration thresholds corresponding to a plurality of detection results of the analyte in the sample to be tested;
obtaining the reaction-complete feature vector of the analyte; and
detecting the reaction-complete feature vector of the analyte based on the plurality of analyte concentration thresholds to obtain the detection result of the analyte in the sample to be tested.

8. The method of claim 7, wherein the reaction-complete feature vector is a reaction-complete feature vector extracted by the signal feature extraction model or a reaction-complete feature vector obtained by performing the time series prediction processing using the time series prediction model.

9. The method of claim 7, further comprising:
adjusting a length of the time window based on a complexity of the fluorescence spectrum data and an identification condition.

10. The method of claim 1, wherein before the time series prediction model predicts the reaction-complete feature vector, the method further comprises:
determining a complexity of the fluorescence spectrum data based on data features of the fluorescence spectrum data;
in response to determining that the complexity satisfies an identification condition, predicting the reaction-complete feature vector by the time series prediction model based on the reaction-in-progress feature vector; and
in response to determining that the complexity does not satisfy the identification condition:
determining a morphological feature vector based on the fluorescence spectrum data corresponding to the reaction in progress; and
predicting the reaction-complete feature vector by the time series prediction model based on the morphological feature vector and the reaction-in-progress feature vector.

11. The method of claim 1, wherein the time series prediction model includes a dual-channel embedding layer and a standard Transformer structure connected in sequence, and predicting the reaction-complete feature vector by the time series prediction model includes:
determining an ideal reaction curve based on test strip parameters;
determining an actual reaction curve based on the fluorescence spectrum data;
determining a residual sequence based on the ideal reaction curve and the actual reaction curve;
inputting the residual sequence and the ideal reaction curve into the dual-channel embedding layer to obtain a residual vector sequence and an ideal feature sequence; and
inputting the residual vector sequence and the ideal feature sequence into the standard Transformer structure to predict the reaction-complete feature vector.

12. The method of claim 1, wherein an output of the detection result output model includes an analyte concentration, and the method further comprises:
generating an electrocardiogram monitoring instruction based on the analyte concentration and sending the electrocardiogram monitoring instruction to an electrocardiogram monitor; and
controlling a recording frequency of the electrocardiogram monitor based on the electrocardiogram monitoring instruction.

13. The method of claim 12, further comprising:
if the analyte concentration satisfies a concentration condition, generating an infusion pump control instruction based on the analyte concentration and physiological parameters of a user, and sending the infusion pump control instruction to a controller of an infusion pump; and
controlling a pumping rate of the infusion pump based on the infusion pump control instruction.

14. The method of claim 12, further comprising:
if the analyte concentration satisfies a concentration condition, generating an unlocking instruction based on the analyte concentration, physiological parameters of a user, and medicine cabinet information, and sending the unlocking instruction to a smart medicine cabinet; and
controlling the smart medicine cabinet to unlock a target drawer based on the unlocking instruction and triggering a prompting device of the target drawer.
